(19) United States
(12) Patent Application Publication
(10) Pub. No.: US 2026/0321404 A1
(43) Pub. Date: Oct. 15, 2026

(54) DISTRIBUTED SYSTEM AND METHOD FOR GENERATING A NURSING SCHEDULE WITH MAXIMUM-MINIMUM FAIRNESS

(71) Applicant: KING SAUD UNIVERSITY, RIYADH (SA)

(72) Inventor: HAMOUD SULTAN BIN OBAID, RIYADH (SA)

(73) Assignee: KING SAUD UNIVERSITY, RIYADH (SA)

(21) Appl. No.: 19/700,209

(22) Filed: Jun. 6, 2026

Related U.S. Application Data
(63) Continuation-in-part of application No. 19/091,779, filed on Mar. 26, 2025.

(51) Int. Cl. G16H 40/20; G06Q 10/0631

(52) U.S. Cl. CPC G16H 40/20; G06Q 10/063116

(57) ABSTRACT

A distributed method for generating and dynamically updating a nursing schedule based on maximum-minimum fairness (MMF) includes executing a scheduling module using one or more networked computing devices communicatively coupled to a central staffing database. The method includes receiving hospital requirements, nurse availability, preferences, and real-time operational data including patient demand signals. A scheduling algorithm is executed in a resource maximizing phase to allocate shift coverage and a load minimizing phase to balance workloads. The method further performs event-driven, incremental schedule recomputation in response to changes in demand or availability while preserving previously assigned constraints. Predicted demand and fairness weighting parameters are incorporated to prioritize critical shifts and promote equitable workload distribution. The resulting schedule is generated and synchronized across computing devices to improve efficiency, responsiveness, and staffing balance.

### CROSS-REFERENCE TO RELATED APPLICATIONS

This application is a continuation-in-part of U.S. patent application Ser. No. 19/091,779, filed on Mar. 26, 2025, the entire contents of which are incorporated herein by reference.

### BACKGROUND

### Field of the Invention

The disclosure of the present patent application relates to resource allocation and computer-implemented scheduling systems, and particularly to a method and system for generating a nursing schedule generated on principles of maximum-minimum fairness.

### Description of Related Art

Effective nurse scheduling is a critical component of healthcare operations, ensuring that hospitals and medical facilities are adequately staffed to meet patient care demands. Traditional nurse scheduling methods often involve processes or heuristic-based approaches that fail to achieve optimal resource allocation. These conventional approaches may lead to overburdened nurses, inefficient shift assignments, and unmet staffing needs, negatively impacting both patient outcomes and employee satisfaction.

Despite the promise of optimization techniques, existing nurse scheduling methods still face several challenges. Many fail to dynamically adapt to real-time constraints such as changing hospital demands, nurse shift preferences, and regulatory work requirements. Additionally, traditional methods do not adequately incorporate mechanisms to fairly distribute workloads among nurses over multiple scheduling periods, which can lead to persistent inequities in shift assignments.

Furthermore, existing scheduling systems are typically implemented as centralized or batch-processing systems that require full recomputation of schedules in response to changing inputs. Such approaches result in increased computational overhead and limited responsiveness to real-time events, including patient admissions, staff availability changes, and cross-department resource demands. Existing systems also lack integration with predictive data sources and do not provide coordinated, distributed scheduling across multiple departments or facilities.

Thus, a computer-implemented system and method of generating and dynamically updating a nursing schedule, capable of operating in a distributed and real-time environment to solve the aforementioned problems is desired.

### SUMMARY

Recent advancements in scheduling techniques have introduced optimization-based solutions to address the inefficiencies of the prior art. One such method is Max-Min Fairness (MMF), which aims to allocate resources and workloads as equitably as possible among available nurses. MMF-based scheduling aims to ensure that nurse availability and workload distribution are balanced optimally to prevent scheduling conflicts and burnout.

The present application utilizes a nurse scheduling method leveraging MMF principles to optimize shift assignments while ensuring fair workload distribution. The method integrates a network-based scheduling framework, allowing hospital administrators, nurses, and other stakeholders to interact through interconnected digital platforms. By utilizing a structured optimization algorithm, the system balances nurse shift allocations in a way that maximizes fairness while adhering to hospital staffing requirements and individual employee preferences.

The present method improves upon existing scheduling methodologies by introducing a structured, iterative approach to identify blocking resources and blocking workloads, enabling a progressive balancing of shift assignments. The algorithm ensures that staffing demands are met efficiently while maintaining equity in workload distribution, ultimately enhancing both hospital operational efficiency and nurse job satisfaction.

The method for nurse scheduling operates within a network framework that interconnects various stakeholders, including hospital administrators, nursing staff, and patient care systems. The multiple stakeholders communicate via a secure hospital intranet, internet, or cloud-based platform. The hospital division system receives inputs for staffing requirements based on patient needs, while nurses submit their shift preferences through an employee portal. A local computer programmed with a scheduling module and algorithm processes these inputs using the scheduling module to generate a fair and efficient schedule. The completed schedule is then distributed to nurses through, for example, text message, email, mobile applications, or web dashboards. Additionally, real-time updates and adjustments can be made dynamically, ensuring flexibility to accommodate last-minute changes or emergencies. The network framework ensures seamless data synchronization, secure access control, and efficient communication among all parties involved in the scheduling process.

The method for generating a nursing schedule includes installing the scheduling module onto a local computing device communicatively coupled over a network to a central staffing database and a plurality of remote computing devices. The method includes receiving, on the local computing device, hospital division requirements, nurse availability, and nurse preferences including real-time operational data comprising at least one of patient admission data, discharge data, or clinical event data received from one or more hospital information systems, to derive input values including:
a set I of nurses, wherein values i in the set of nurses include names of individual nurses;
a set L of nurse levels associated with the set of nurses, wherein the values l in the set of nurse levels indicate if a nurse is a senior level nurse;
a two-dimensional set IL of each nurse and their level;
a set of preferences associated with the set of nurses, wherein the set of preferences associated with the set of nurses include shift preferences of weekday, weekend, day, and night;
a set W of weeks, wherein values w in the set of weeks include week-long time periods on a calendar;
a set D of days, wherein values in the set of days indicate different days d of a week; a two-dimensional set WD of weeks and days;
a set S of shifts per day, wherein the set of shifts per day include a number of shifts s required for a calendar day;
a number MB of maximum beds per day per shift;
a number WR of maximum regular shifts per nurse per week;
a number MR of maximum total regular shifts per nurse per month;
a number WO of maximum total overtime shifts per nurse per week; and a number MO of maximum total overtime shifts per nurse per month.

The method continues by executing on the local computing device a scheduling algorithm on the scheduling module configured to perform event-driven and incremental schedule recomputation in response to detected changes in real-time operational data. The scheduling algorithm includes an initialization stage followed by a resource maximizing phase followed by a load minimizing phase. The initialization stage includes setting n, m, θ, δ_{sd}, λ_{sd} and σ_{i,j} to 0 and γᵢₗ to M, wherein n and m are iteration counters, θ is a target load parameter used in deviation minimization, δ_{sd} is a binary parameter indicating whether a total shift allocation is maximized, λ_{sd} is a lower bound on a number of shifts assigned, σ_{i,l} is a binary parameter indicating whether a nurse load is minimized, γᵢₗ is an upper bound on a number of shifts assigned, and M is a sufficiently large number greater than 500 and preferably greater than 1000;

After the initialization stage, the algorithm continues to the resource maximizing phase, which includes: a first step of determining if all available shifts have been maximized in which Σ_{s∈S}Σ_{i∈I}δ_{sd}=|S∥D| and if so, ending the maximizing phase, and if not, setting n=n+1 and proceeding to maximize all available shifts within the bounds and parameters through solving an objective function P_{R}=αₙ using a constraint

$${\sum }_{i , l ∈ IL} {x}_{il}^{swd} + {y}_{il}^{swd} \geq {α}_{n} - M {δ}_{sd}$$

for P_{R}(θ, δ, λ, σ, γ) in which αₙ is a current level of shift allocation being maximized.

A second step in the resource maximizing phase includes setting λ_{sd}=αₙ at iteration n to progressively increase shift assignments and prevent optimized shift allocations from being reduced in later iterations. A third step includes setting θ=αₙ+ε, and solving an objective function

$${P}_{SND} = {\sum }_{s ∈ S} {\sum }_{d ∈ D} {U}_{sd} ( 1 - {δ}_{sd} ) {d}_{sd}^{-}$$

for P_{SND}(θ, δ, λ, σ, γ) to minimize a sum of negative deviations (P_{SND}) in which ε is a number less than 1,

$${d}_{sd}^{-}$$

is a negative deviation from a total number of shifts for shift s on day d., and U_{sd} is a parameter representing the weight (urgency) of the shift s on day d. A fourth step includes setting

$${δ}_{sd} = 1 if {d}_{sd}^{-}$$

fails to be minimized to zero. The first, second, third and fourth steps are repeated until all total shifts are blocking. The number of nurses assigned per day and shift is limited by the number MB of maximum beds per day per shift as defined by a constraint

$${\sum }_{i , l ∈ IL} {x}_{il}^{swd} + {y}_{il}^{swd} \leq MB ,$$

where x and y are binary decision variables, and once all shifts are blocking, proceeding to the load minimizing phase.

The load minimizing phase includes a fifth step of determining if Σ_{i,l∈IL}σᵢₗ=|I∥L| and if not, setting m=m+1 and finding a minimum βₘ by solving an objective function P_{L}=βₘ for P_{L}(θ, δ, λ, σ, γ) where βₘ is an upper bound on a number of loads assigned to each nurse at iteration m. A sixth step includes setting γᵢₗ=βₘ if σᵢₗ=0. A seventh step includes setting θ=βₘ−ε and solving an objective function

$${P}_{SPD} = {\sum }_{( i , l ) ∈ IL} ( 1 - {σ}_{il} ) {d}_{il}^{+}$$

for P_{SPD}(θ, δ, λ, σ, γ) to minimize a weighted sum of positive deviations (P_{SPD}) where

$${\hat{d}}_{il}^{+}$$

is a positive deviation from a total load of each nurse i of level l and Pᵢₗ is a parameter representing the weight (points) of the nurse i of level l. An eighth step includes determining if a load of a nurse i of level l is blocking by checking if

$${d}_{il}^{+} > 0$$

and if so, setting δᵢₗ=1 to designate the load is blocking. The fifth, sixth, seventh, and eighth, steps are repeated until all nurse loads are blocking (i.e. cannot be further optimized). The central staffing database is updated with the recomputed schedule while synchronizing schedule changes with one or more remote computing devices in substantially real-time. The method concludes by converting the raw numerical data of the optimized resources and loads into a human-readable completed schedule to be distributed. The raw numerical data is converted by extracting, by the scheduling module, numerical shift allocation data generated by the scheduling algorithm, converting the numerical shift allocation data into a structured schedule matrix stored in memory of the local computing device, and automatically generating, from the structured schedule matrix, a machine-generated timetable representing nurse shift assignments. The completed schedule may be in the form of a timetable comprising rows and columns and/or in the form of a timetable comprising color-coding or shading, wherein the color-coding or shading indicates shift assignments for nurses.

Once the completed schedule is formed, the method may continue by electronically sending the completed schedule through the local computing device to one or more remote computing devices (such as through email and/or text message or the like) of hospital staff members. The method for generating a nursing schedule may use hospital division requirements such as bed capacity, nurse shift length, required work hours per week, maximum overtime hours per week, and total days and weeks of schedule to derive the scheduling input values.

A constraint

$${\sum }_{s ∈ S} {\sum }_{d ∈ D} {x}_{il}^{swd} \leq MR$$

may be used in the scheduling method to ensure that each nurse does not exceed the number MR of maximum total regular shifts per nurse per month.

A constraint

$${\sum }_{s ∈ S} {\sum }_{d ∈ D} {y}_{il}^{swd} \leq MO$$

may be used in the scheduling method to ensure that each nurse does not exceed the number MO of maximum total overtime shifts per nurse per month.

A constraint

$${\sum }_{d \vert ( w , d ) ∈ WD} {\sum }_{s ∈ S} {x}_{il}^{swd} \leq WS$$

may be used in the scheduling method to ensure that each nurse does not exceed a number WS of regular and overtime shifts each week. The number WS of regular and overtime shifts each week is derived from the hospital division requirements.

A constraint

$$1 - {x}_{il}^{2 - a , wd} - {y}_{il}^{2 - a , wd} \geq {x}_{il}^{1 + a , wd + b} + {y}_{il}^{1 + a , wd + b}$$

may be used in the scheduling method to prevent switching a nurse from a day shift to a night shift until the nurse takes two days off given that parameter a switches shifts, and parameter b applies the constraint on the next day.

A constraint

$${\sum }_{s ∈ S} {x}_{il}^{swd} + {y}_{il}^{swd} \leq 1$$

may be used in the scheduling method to prevent assigning a regular shift and an overtime shift to each nurse simultaneously.

A constraint

$$4 - {\sum }_{k = d}^{d + 4} {\sum }_{s ∈ S} {x}_{il}^{swk} + {y}_{il}^{swk} \geq {x}_{il}^{swd + 4} + {y}_{il}^{swd + 4}$$

may be used in the scheduling method to set a limit on a number of consecutive working days to be four days, after which a nurse is required to take at least one day off.

A constraint

$${x}_{il}^{swd} + {y}_{il}^{swd} \geq 1$$

may be used in the scheduling method to assign at least one senior level nurse in every shift.

A constraint

$${\sum }_{s ∈ S} {x}_{il}^{swd} + {y}_{il}^{swd} \leq {A}_{id}$$

may be used in the scheduling method to stop assigning any load to a nurse i if they are not available, in which A_{id} is a parameter that equals 1 if a nurse i is available to work on day d and 0 otherwise.

A constraint

$${\sum }_{i , l ∈ IL} {x}_{il}^{swd} + {y}_{il}^{swd} - θ + {d}_{swd}^{-} - {d}_{swd}^{+} = 0$$

may be used in the scheduling method to ensure that a total number of assigned shifts for nurse i on a day d of week w remains as close as possible to the target load parameter θ.

A constraint

$${\sum }_{i , l ∈ IL} {U}_{sd} ( {x}_{il}^{swd} + {y}_{il}^{swd} ) - {λ}_{swd} \geq 0$$

may be used in the scheduling method to set a lower bound for a number of shifts assigned.

A constraint

$${\sum }_{s ∈ S} {\sum }_{( w , d ) ∈ WD} {P}_{il} ( {x}_{il}^{swd} + {y}_{il}^{swd} ) \leq {β}_{n} + M {σ}_{il}$$

may be used in the scheduling method to dynamically regulate how many shifts a nurse can be assigned, enforcing fairness until loads reach a blocking state where the loads can no longer be reduced.

A constraint

$${\sum }_{s ∈ S} {\sum }_{( w , d ) ∈ WD} {x}_{il}^{swd} + {y}_{il}^{swd} - θ + {\hat{d}}_{il}^{-} - {\hat{d}}_{il}^{+} = 0$$

may be used in the scheduling method to enforce fairness by ensuring each nurse i is assigned a workload close to the target parameter θ, allowing for small positive or negative deviations to maintain feasibility in scheduling.

A constraint

$${\sum }_{s ∈ S} {\sum }_{( w , d ) ∈ WD} {P}_{il} ( {x}_{il}^{swd} + {y}_{il}^{swd} ) - {γ}_{il} \leq 0$$

may be used in the scheduling method to prevent any nurse from being overburdened by limiting total assigned shifts to be no greater than γᵢₗ.

A constraint

$${x}_{il}^{swd} , {y}_{il}^{swd} ∈ \{ 0 , 1 \}$$

may be used in the scheduling method to enforce a binary yes or no decision for assigning nurses to shifts, ensuring that partial assignments are not allowed.

A constraint

$${d}_{swd}^{-} , {d}_{swd}^{+} \geq 0$$

may be used by the scheduling method to ensure that imbalances in shift assignments are measured as a non-negative deviation thereby preventing cancellation effects where an excess of shifts offsets a shortage of shifts.

A constraint

$${\hat{d}}_{il}^{-} , {\hat{d}}_{il}^{+} \geq 0$$

may be used in the scheduling method to track and correct any unfair distribution of shifts among nurses by ensure deviations are positive whereby imbalances can be quantified and minimized, ensuring that no nurse is overburdened or underutilized unfairly.

These and other features of the present subject matter will become readily apparent upon further review of the following specification.

### BRIEF DESCRIPTION OF DRAWINGS

FIG. 1 is a diagram representation of stakeholders involved in a method of generating and distributing a nursing schedule.

FIG. 2 is a diagram representation of certain hardware elements used in a method of generating and distributing a nursing schedule.

FIG. 3A is a block diagram of a scheduling module and algorithm based on maximum-minimum fairness.

FIG. 3B is a block diagram of a method of converting raw numerical output from a scheduling algorithm into a human-readable schedule.

FIG. 3C are examples of human-readable schedules.

FIG. 4 is a diagram illustrating a distributed scheduling system architecture including a plurality of departmental scheduling optimizer nodes in communication with a central staffing database and one or more external data sources.

FIG. 5 is a diagram illustrating an incremental scheduling timeline showing selectively modifiable schedule regions and user response-dependent schedule locking.

FIG. 6A is a chart of total resource utilization using a first scheduling model according to the present teachings in a first scenario.

FIG. 6B is a chart of total resource utilization using a second model in a first scenario.

FIG. 6C is a chart of total resource utilization using a third model in a first scenario.

FIG. 6D is a chart of regular and overtime resource utilization using a scheduling model according to the present teachings in a first scenario.

FIG. 6E is a chart of regular and overtime resource utilization using a second model in a first scenario.

FIG. 6F is a chart of regular and overtime resource utilization using a third model in a first scenario.

FIG. 6G is a chart of total load distribution using a first scheduling model according to the present teachings in a first scenario.

FIG. 6H is a chart of total load distribution using a second model in a first scenario.

FIG. 6I is a chart of total load distribution using a third model in a first scenario.

FIG. 7A is a chart of total resource utilization using a first scheduling model according to the present teachings in a second scenario.

FIG. 7B is a chart of total resource utilization using a second model in a second scenario.

FIG. 7C is a chart of total resource utilization using a third model in a second scenario.

FIG. 7D is a chart of regular and overtime resource utilization using a scheduling model according to the present teachings in a second scenario.

FIG. 7E is a chart of regular and overtime resource utilization using a second model in a second scenario.

FIG. 7F is a chart of regular and overtime resource utilization using a third model in a second scenario.

FIG. 7G is a chart of total load distribution using a first scheduling model according to the present teachings in a second scenario.

FIG. 7H is a chart of total load distribution using a second model in a second scenario.

FIG. 7I is a chart of total load distribution using a third model in a second scenario.

Similar reference characters denote corresponding features consistently throughout the attached drawings.

### DETAILED DESCRIPTION

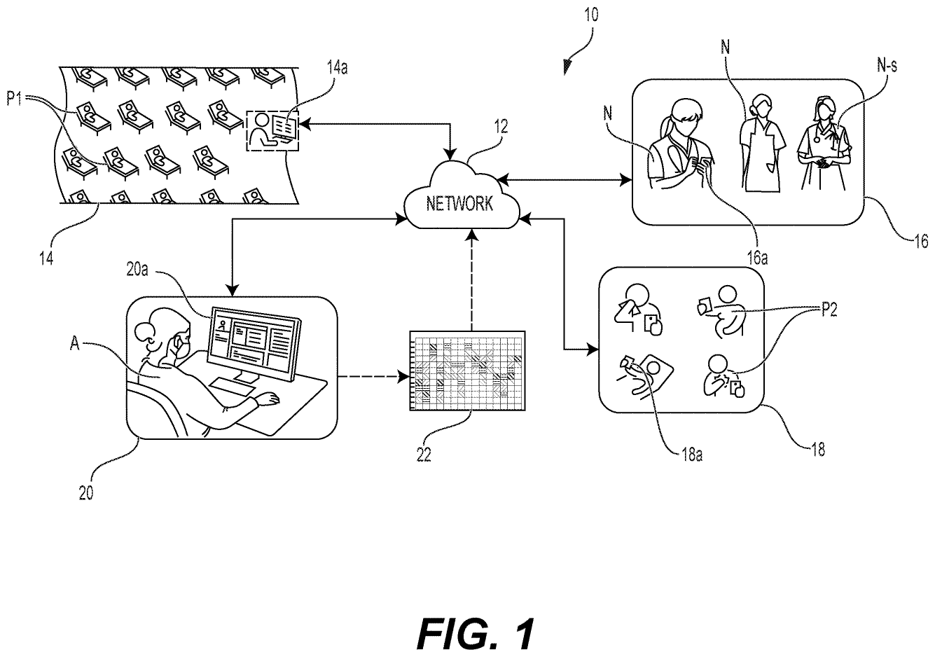

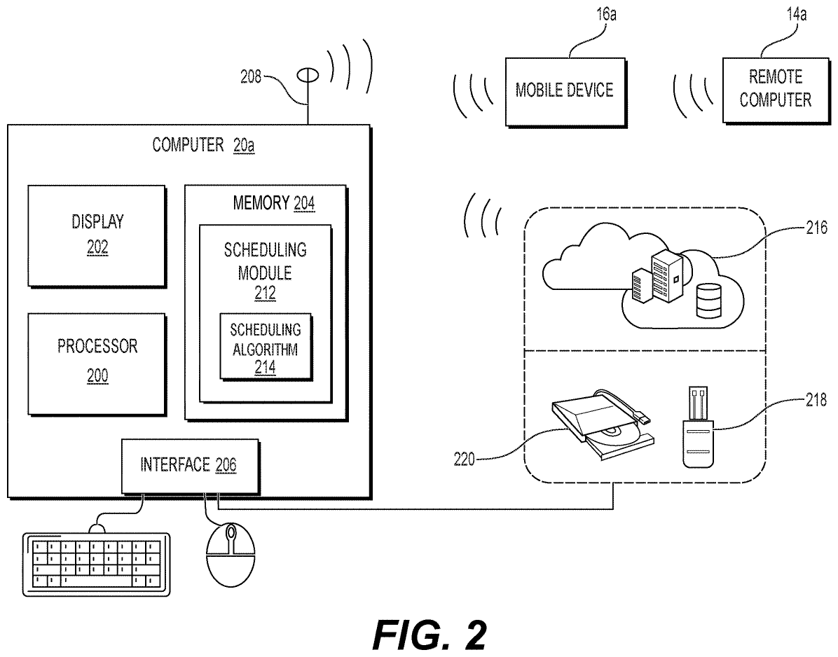

Referring to FIG. 1, system 10 is composed of a network 12 that links all the stakeholders involved in the nurse scheduling process, including hospital division 14, employee system 16, patient system 18, and scheduling engine 20. The nurse scheduling process starts with a hospital division 14 entering the division's requirements, including number of nurses needed based on the number of patients P1, through a remote computer 14a of the hospital division system 14. Next, nurses N of employee system 16 enter their preferences, including day or night shift, weekday or weekend, using a remote computer in the form of, for example, a mobile device 16a (or other remote computer such as a laptop, pc, etc.). It should be noted that the term “system” as used in reference to FIG. 1 may refer to an interface portal used on a computing device by the entities of the system to submit information. Employee system 16 is also used to supply information regarding a total number of available nurses, and seniority levels of each nurse, in which senior level nurses N-s are indicated. The nurse preferences may be given a cumulative weight if a nurse does not receive their preferences in order to be given a higher priority in a subsequently generated schedule. Patient system 18 may provide information regarding new incoming patients P2 using remote computing devices 18a. Patient system 18 may also serve as a portal for communication between hospital staff and patients. A scheduling engine 20 receives the resource, load, and scheduling requirement information from the hospital division 14, employee system 16, and patient system 18 on a local computer 20a which is programmed with a scheduling model (to be discussed more in FIG. 2) to generate a completed nursing schedule 22. Optionally, a scheduling administrator A may also enter pieces of information, if needed, into the scheduling model. The nursing schedule 22 is sent from the local computer 20a to one or more remote computing devices over the network 12, such as hospital division remote computer 14a and employee remote computer 16a. While scheduling engine 20 is depicted as using a local computer 20a, it should be understood that any type of computer may be used by scheduling engine 20, such as a central server or the like running an automated process. The automated process may run automatically, for example, every scheduling period as determined by the users of the system.

FIG. 2 shows a diagram of certain hardware elements of the disclosed scheduling method including an example local computer 20a used by the scheduling engine 20 of FIG. 1. In a basic configuration, computer 20a may include components such as a processor 200, display 202, memory 204, interface 206, and wireless transmitter 208. Examples of the processor 200 may include, but are not limited to, a general-purpose processor, a central processing unit (CPU), a microprocessor, a digital signal processor (DSP), a controller, a microcontroller, a state machine, a programmable logic controller, logical control circuitry or the like. The memory 204 may include, but is not limited to, the following non-transitory computer readable media: random access memory (RAM), read-only memory (ROM), non-volatile random access memory (NVRAM), programmable read-only memory (PROM), erasable programmable read-only memory (EPROM), electrically erasable PROM (EEPROM), flash memory, magnetic or optical data storage, or any other suitable type of non-transitory media.

Interface 206 may be any suitable type of interface allowing the system components to operate, and may include, but are not limited to, a combination of buttons, switches, touchscreens, keyboards or the like. Display 202 may be any necessary displays or other types of visual indicators for operators of the system, including, but not limited to, computer monitor displays, LED displays, liquid crystal displays, touchscreen displays and the like. One or more wireless transmitters 208 may be included for transmission of 2.4 Ghz and 5 Ghz wi-fi, Bluetooth transmission, satellite transmission, and/or radio frequency (RF) transmission.

As is known, mobile device 16a used by nurses N of FIG. 1 and remote computer 14a used by hospital division 14 of FIG. 1 may include the same or similar hardware components as computer 20a including processors, memory, display, interfaces, wireless transmitters/receivers and the like.

In addition to the basic components such as a processor 200, display 202, memory 204, and interface 206, and wireless transmitter 208, computer 20a includes a scheduling module 212 with an embedded scheduling algorithm 214 programmed into memory 204. Scheduling module 212 may be programmed onto computer 20a from any suitable source such as an internet web download 216, from a removable storage device 218, or from a program disk 220. In addition, scheduling module 212 may be preinstalled on computer 20a. A detailed explanation of the scheduling algorithm and scheduling module follows directly below.

### Scheduling Algorithm and Experimentation

### Definitions

It is noted that, as used in this specification and the appended claims, certain terms are used in the following manner as described.

“Blocking” refers to a resource or load that may not be optimized further.

“Maximize” or “Maximized” refers to the process of increasing a variable to its highest possible value within given bounds, parameters and constraints.

“Minimize” or “Minimized” refers to the process of decreasing a variable to its lowest possible value withing given bounds, parameters and constraints.

“Set” refers to a collection of related values that are grouped based on a category or criteria.

“Resource” or “Resources” unless otherwise indicated (e.g. as referring to an employee or nurse) refers to a total number of shifts needing to be allocated, including weekday, weekend, regular, and overtime shifts.

“Load” refers to a total workload assigned to each nurse, typically measured in amount of assigned shifts, but potentially also measured in days worked, hours worked, etc.

“Bound” refers to a limit value placed on a variable.

“Parameter” refers to a value used to guide optimization.

“Constraint” refers to a rule or condition that must be satisfied.

“Variable” refers to a quantity that can change or be optimized.

“Decision variable” refers to a key value used in optimization and may indicate whether or not certain conditions hold true.

“Objective function” refers to a formula to be optimized.

“Maximum-Minimum Fairness (or, MMF)” generally refers to a set of fairness criterions to ensure equitable and fair distribution.

“Double direct progressive” generally refers to a two-phase iterative process for optimization.

The following disclosure details the development of a scheduling approach based on a self-described double direct progressive filling algorithm (DDPFA) which aims to find the maximum-minimum fairness (MMF) demand shifts and the MMF nurse loads. The algorithm maximizes the total demand shifts (regular, overtime, weekend, weekday) until a set of rules, such as nurse availability, shift limits, or hospital constraints, reach their maximum allowable limits and prevent further allocation of shifts. The scheduling algorithm is capable of detecting blocking resources and removing them from the process in the next iteration. This process is repeated until all resources have reached their maximum allowable limit, at which point the resources are considered to be at maximum-minimum fairness. In the second phase, after maximizing the total assigned shifts, the algorithm seeks to fairly distribute the workload among available nurses, with the goal of minimizing the total load per nurse while ensuring fairness. The algorithm keeps reducing individual nurse workloads until some nurses reach a minimum feasible limit, meaning that their schedules cannot be reduced further without violating constraints (e.g., ensuring minimum staffing levels). These nurses with non-reducible workloads are considered blocking loads because they prevent further minimization of workload within the system constraints. Once all remaining workloads are blocking, the process stops, resulting in a maximum-minimum fair distribution of workload across all nurses.

The algorithm relies on a mixed integer linear programming (MILP) model including four objective functions. The sets, parameters, decision variables, objective functions, and constraints are discussed in the following paragraphs.

### Sets and Parameters

I is the set of nurses.
L is the set of nurse levels.
IL is a two-dimensional set of each nurse and their level.
W is the set of weeks.
D is the set of days.
WD is a two-dimensional set of weeks and days.
S is the set of shifts per day.
MB is the Maximum number beds per day per shift.
WR is the Maximum regular shifts per nurse per week.
WO is the Maximum overtime shifts per nurse per week.
MR is the Maximum total regular shifts per nurse per month.
MO is the Maximum total overtime shifts per nurse per month.
δ_{sd} is a binary parameter that equals 1 if the number of nurses in shift s on day d is blocking, and 0 otherwise.
σᵢₗ is a binary parameter that equals 1 if the number of shifts of nurse i of level l is blocking, and 0 otherwise.
λ_{sd} is a minimum bound of the number of nurses in shift s on day d.
γᵢₗ is a maximum bound of the number of shifts of nurse i of level l.
M is a sufficiently large number.
ε is a sufficiently small number.
U_{sd} is a parameter representing the weight (urgency) of the shift s on day d
Pᵢₗ is a parameter representing the weight (points) of the nurse i of level l
A_{id} is a parameter that equals 1 if nurse i is available to work in day d, and 0 otherwise

### Decision Variables

$${x}_{il}^{swd} = 1$$

if nurse i with level l is scheduled to work a regular shift in shift s in week w on day d, and 0 otherwise.

$${y}_{il}^{swd} = 1$$

if nurse i with level l is scheduled to work an overtime shift in shift s in week w on day d, and 0 otherwise.

$${d}_{swd}^{-} = the negative deviation from total number of shifts for shift s in week w on day d .$$

$${d}_{swd}^{+} = the positive deviation from total number of shifts for shift s in week w on day d .$$

$${\hat{d}}_{il}^{-} = the negative deviation from total load for each nurse i and their level l .$$

$${\hat{d}}_{il}^{+} = the positive deviation from total load for each nurse i and their level l .$$

$${α}_{n} = the lower bound on the number nurses assigned in each day and each shift at iteration n .$$

βₘ=the upper bound on the number of loads assigned to each nurse at the iteration m.

### Objective Functions

| Number | Objective Function |
|---|---|
| (1) | Max P_{R} = αₙ |
| (2) | Min P_{L} = βₘ |
| (3) | $Min {P}_{SND} = {\sum }_{s ∈ S} {\sum }_{d ∈ D} ( 1 - {δ}_{sd} ) {U}_{sd} {d}_{sd}^{-}$ |
| (4) | $Min {P}_{SPD} = {\sum }_{( i , l ) ∈ IL} ( 1 - {σ}_{il} ) {P}_{il} {d}_{il}^{+}$ |

### Constraints

| Number | Constraint | Condition |
|---|---|---|
| (5) | ${\sum }_{i , l ∈ IL} {x}_{il}^{swd} + {y}_{il}^{swd} \leq MB$ | ∀ s ∈ S, (w, d) ∈ WD |
| (6) | ${\sum }_{s ∈ S} {\sum }_{d ∈ D} {x}_{il}^{swd} \leq MR$ | ∀ (i, l) ∈ IL |
| (7) | ${\sum }_{s ∈ S} {\sum }_{d ∈ D} {y}_{il}^{swd} \leq MO$ | ∀ (i, l) ∈ IL |
| (8) | ${\sum }_{d \vert ( w , d ) ∈ WD} {\sum }_{s ∈ S} {x}_{il}^{swd} \leq WS$ | ∀ (i, l) ∈ IL, w ∈ W |
| (9) | ${\sum }_{d \vert ( w , d ) ∈ WD} {\sum }_{s ∈ S} {x}_{il}^{swd} \leq WS$ | ∀ (i, l) ∈ IL, w ∈ W |
| (10) | $1 - {x}_{il}^{2 - a , wd} - {y}_{il}^{2 - a , wd} \geq {x}_{il}^{1 + a , wd + b} + {y}_{il}^{1 + a , wd + b}$ | ∀a ∈ {0, 1}, b ∈ {1, 2}, (i, l) ∈ |
| | | IL, (w, d) ∈ WD\|d ≤ |
| | | \|D\| − b |
| (11) | ${\sum }_{s ∈ s} {x}_{il}^{swd} + {y}_{il}^{swd} \leq 1$ | ∀(i, l) ∈ IL, (w, d) ∈ WD |
| (12) | $4 - {\sum }_{k = d}^{d + 4} {\sum }_{s ∈ S} {x}_{il}^{swk} + {y}_{il}^{swk} \geq {x}_{il}^{swd + 4} + {y}_{il}^{swd + 4}$ | ∀(i, l) ∈ IL, d ∈ D\|(w,d) ∈ WD, d ≤ 24 |
| (13) | ${x}_{il}^{swd} + {y}_{il}^{swd} \geq 1$ | ∀(i, l) ∈ IL\|l = 1, s ∈ |
| | | S, (w, d) ∈ WD |
| (14) | ${\sum }_{s ∈ s} {x}_{il}^{swd} + {y}_{il}^{swd} \leq {A}_{id}$ | ∀(i, l) ∈ IL, (w, d) ∈ WD |
| (15) | ${\sum }_{i , l ∈ IL} {U}_{sd} ( {x}_{il}^{swd} + {y}_{il}^{swd} ) \geq {α}_{n} - M {δ}_{sd}$ | ∀s ∈ S, (w, d) ∈ WD |
| (16) | ${\sum }_{i , l ∈ IL} {x}_{il}^{swd} + {y}_{il}^{swd} - θ + {d}_{swd}^{-} - {d}_{swd}^{+} = 0$ | ∀ s ∈ S, (w, d) ∈ WD |
| (17) | ${\sum }_{i , l ∈ IL} {U}_{sd} ( {x}_{il}^{swd} + {y}_{il}^{swd} ) - {λ}_{swd} \geq 0$ | ∀ s ∈ S, (w, d) ∈ WD |
| (18) | ${\sum }_{s ∈ S} {\sum }_{d \vert ( w , d ) ∈ WD} {P}_{il} ( {x}_{il}^{swd} + {y}_{il}^{swd} ) \leq {β}_{n} + M {σ}_{il}$ | ∀ (i, l) ∈ IL |
| (19) | ${\sum }_{s ∈ S} {\sum }_{d \vert ( w , d ) ∈ WD} {x}_{il}^{swd} + {y}_{il}^{swd} - θ + {\hat{d}}_{il}^{-} - {\hat{d}}_{il}^{+} = 0$ | ∀ (i, l) ∈ IL |
| (20) | ${\sum }_{s ∈ S} {\sum }_{d ∈ D} {P}_{il} ( {x}_{il}^{swd} + {y}_{il}^{swd} ) - {γ}_{il} \leq 0$ | ∀ (i, l) ∈ IL |
| (21) | ${x}_{il}^{swd} , {y}_{il}^{swd} ∈ \{ 0 , 1 \}$ | ∀ (i, l) ∈ IL, s ∈ |
| | | S, (w, d) ∈ WD |
| (22) | ${d}_{swd}^{-} , {d}_{swd}^{+} \geq 0$ | ∀ s ∈ S, (w, d) ∈ WD |
| (23) | ${\hat{d}}_{il}^{-} , {\hat{d}}_{il}^{+} \geq 0$ | ∀ (i, l) ∈ IL |

The first objective function

$$Max {P}_{R} = {α}_{n} \qquad ( 1 )$$

maximizes the minimum total nurses for each day d and shift s, and the second objective function

$$Min {P}_{L} = {β}_{m} \qquad ( 2 )$$

minimizes the maximum number of shifts assigned to each nurse. The objective function

$$Min {P}_{SND} = {\sum }_{s ∈ S} {\sum }_{d ∈ D} ( 1 - {δ}_{s d} ) {U}_{s d} {d}_{s d}^{-} \qquad ( 3 )$$

minimizes the weighted sum of negative deviations (i.e. variation from the target value) from required shift allocations, wherein the weighting factor U_{sd} represents an urgency weight or demand-based importance of shift s on day d such that deviations in higher-demand shifts are prioritized for reduction. The objective function

$$Min {P}_{SPD} = {\sum }_{( i , l ) ∈ IL} ( 1 - {σ}_{i l} ) {P}_{i l} {d}_{i l}^{+} \qquad ( 4 )$$

minimizes the weighted sum of positive deviations from target nurse workloads, wherein the weighting factor Pᵢₗ represents a priority or fairness weight associated with nurse i of level l, such that workload imbalances are reduced in accordance with fairness or incentive considerations. The number of nurses assigned per day and shift is limited by the number of beds available as seen in constraint

$${\sum }_{i , l ∈ IL} {x}_{i l}^{s w d} + {y}_{i l}^{s w d} \leq MB . \qquad ( 5 )$$

The constraint

$${\sum }_{s ∈ S} {\sum }_{d ∈ D} {x}_{i l}^{s w d} \leq MR \qquad ( 6 )$$

ensures that each nurse does not exceed the monthly regular shift limit. The constraint

$${\sum }_{s ∈ S} {\sum }_{d \vert ( w , d ) ∈ WD} {y}_{i l}^{s w d} \leq MO \qquad ( 7 )$$

imposes a limit on the monthly total number of overtime shifts for each nurse. Constraints

$${\sum }_{d \vert ( w , d ) ∈ WD} {\sum }_{s ∈ S} {x}_{i l}^{s w d} \leq WS \qquad ( 8 )$$

and

$${\sum }_{d \vert ( w , d ) ∈ WD} {\sum }_{s ∈ S} {x}_{i l}^{s w d} \leq WS \qquad ( 9 )$$

are imposed to comply with the hospital policy that the nurse has a limit of the regular or overtime shifts each week. The constraint

$$1 - {x}_{i l}^{2 - a , w d} - {y}_{i l}^{2 - a , w d} \geq {x}_{i l}^{1 + a , w d + b} + {y}_{i l}^{1 + a , w d + b} \qquad ( 10 )$$

does not allow switching a nurse from a day shift to a night shift (the opposite is true) until they take two days off given that the parameter “a” switches shifts, and parameter “b” applies the constraint on the next day and the day that follows. The constraint

$${\sum }_{s ∈ S} {x}_{i l}^{s w d} + {y}_{i l}^{s w d} \leq 1 \qquad ( 11 )$$

prevents assigning a regular shift and an overtime shift to each nurse simultaneously. The constraint

$$4 - {\sum }_{k = d}^{d + 4} {\sum }_{s ∈ S} {x}_{i l}^{s w k} + {y}_{i l}^{s w k} \geq {x}_{i l}^{s w d + 4} + {y}_{i l}^{s w d + 4} \qquad ( 12 )$$

sets a limit on the number of consecutive working days to be four days, after which a nurse has to take at least one day off. The constraint

$${x}_{i l}^{s w d} + {y}_{i l}^{s w d} \geq 1 \qquad ( 13 )$$

assigns at least one in-charge nurse in every shift. The constraint

$${\sum }_{s ∈ S} {x}_{i l}^{s w d} + {y}_{i l}^{s w d} \leq {A}_{id} \qquad ( 14 )$$

stops assigning any load to nurse i if they are not available. The total number of daily shifts is maximized through αₙ, in the objective function

$$Max {P}_{R} = {α}_{n} \qquad ( 1 )$$

an using the constraint

$${\sum }_{i , l ∈ IL} {U}_{sd} ( {x}_{il}^{swd} + {y}_{il}^{swd} ) \geq {α}_{n} - M {δ}_{sd} . \qquad ( 15 )$$

The constraint

$${\sum }_{i , l ∈ IL} {x}_{il}^{swd} + {y}_{il}^{swd} - θ + {d}_{swd}^{-} - {d}_{swd}^{+} = 0 \qquad ( 16 )$$

is a goal constraint used to identify blocking shifts. A lower bound is set for the blocking shifts by constrain

$${\sum }_{i , l ∈ IL} {U}_{sd} ( {x}_{il}^{swd} + {y}_{il}^{swd} ) - {λ}_{swd} \geq 0. \qquad ( 17 )$$

The decision variable βₘ minimizes the total load for each nurse through the constraint

$${\sum }_{s ∈ S} {\sum }_{d ∈ D} {P}_{il} ( {x}_{il}^{swd} + {y}_{il}^{swd} ) \leq {β}_{n} + M {σ}_{il} \qquad ( 18 )$$

and the objective function

$$Min {P}_{L} = {β}_{m} . \qquad ( 2 )$$

The goal constraint

$${\sum }_{s ∈ S} {\sum }_{d \vert ( w , d ) ∈ WD} {x}_{il}^{swd} + {y}_{il}^{swd} - θ + {\hat{d}}_{il}^{-} - {\hat{d}}_{il}^{+} = 0 \qquad ( 19 )$$

identifies the blocking shifts, and the set of blocking shifts are given an upper bound using the constraint

$${\sum }_{s ∈ S} {\sum }_{d \vert ( w , d ) ∈ WD} {P}_{il} ( {x}_{il}^{swd} + {y}_{il}^{swd} ) - {γ}_{il} \leq 0. \qquad ( 20 )$$

The constraint

$${x}_{il}^{swd} , {y}_{il}^{swd} ∈ \{ 0 , 1 \} \qquad ( 21 )$$

enforces a binary yes or no decision for assigning nurses to shifts, ensuring that partial assignments are not allowed. The constraint

$${d}_{swd}^{-} , {d}_{swd}^{+} \geq 0 \qquad ( 22 )$$

ensures that imbalances in shift assignments are measured as a non-negative deviation thereby preventing cancellation effects where an excess of shifts offsets a shortage of shifts. The constraint

$${\hat{d}}_{il}^{-} , {\hat{d}}_{il}^{+} \geq 0 \qquad ( 23 )$$

is used to track and correct any unfair distribution of shifts among nurses by ensuring deviations are positive whereby imbalances can be quantified and minimized, ensuring that no nurse is overburdened or underutilized unfairly.

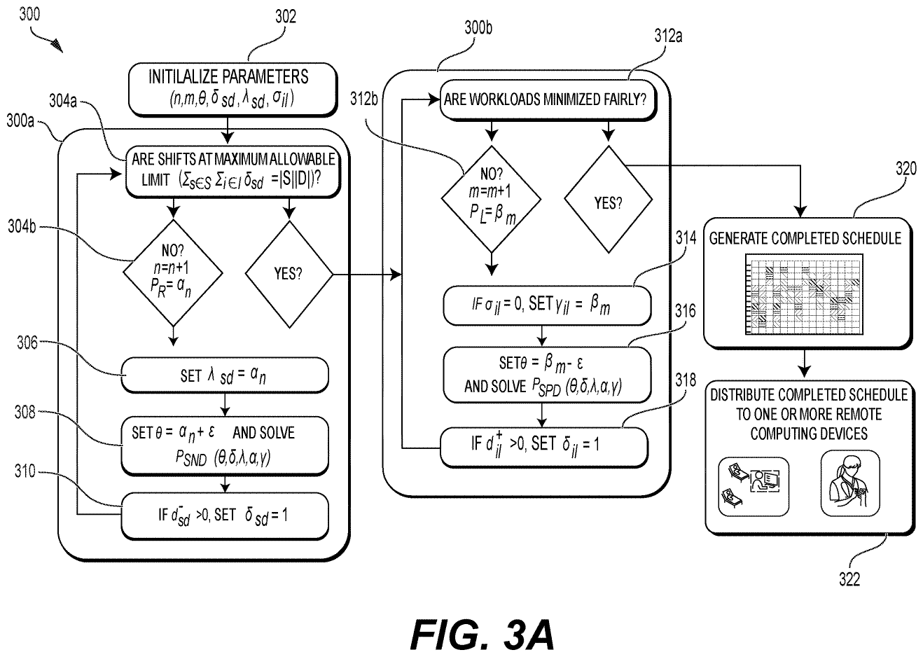

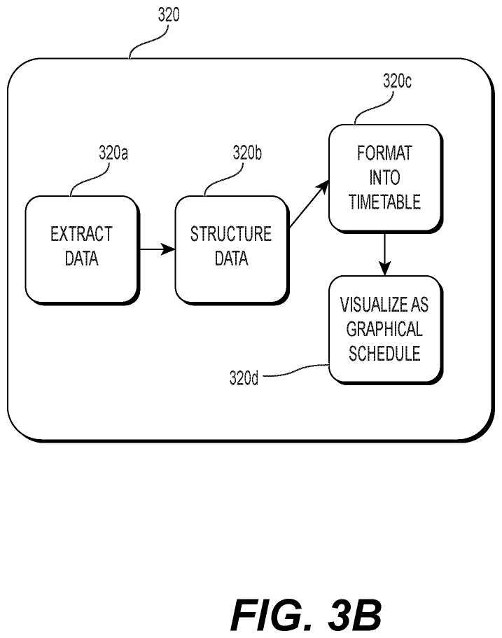

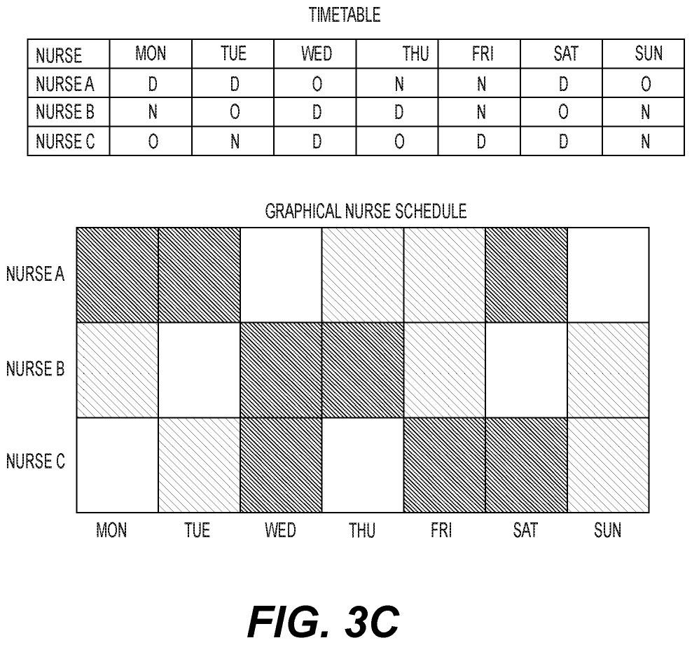

With reference now to FIG. 3A (and limited reference to FIGS. 1-2), the method for generating nursing schedules includes installing a scheduling module 212 (FIG. 2) onto a local computing device 20a and receiving, on the local computing device 20a, requirements of hospital division 14 (FIG. 1) such as bed capacity, nurse shift length, required work hours per week, maximum overtime hours per week, and total days and weeks of a schedule to derive scheduling input values. Employee system 16 supplies information regarding nurse availability and nurse preferences. The information from hospital division 14 and employee system 16 are supplied to the scheduling engine 20 and used to derive input values including:
a set I of nurses, wherein values i in the set of nurses include names of individual nurses;
a set L of nurse levels associated with the set of nurses, wherein the values l in the set of nurse levels indicate if a nurse is a senior level nurse;
a two-dimensional set IL of each nurse and their level;
a set of preferences associated with the set of nurses, wherein the set of preferences associated with the set of nurses include shift preferences of weekday, weekend, day, and night;
a set W of weeks, wherein values w in the set of weeks include week-long time periods on a calendar;
a set D of days, wherein values in the set of days indicate different days d of a week;
a two-dimensional set WD of weeks and days;
a set S of shifts per day, wherein the set of shifts per day include a number of shifts s required for a calendar day;
a number MB of maximum beds per day per shift;
a number WR of maximum regular shifts per nurse per week;
a number MR of maximum total regular shifts per nurse per month;
a number WO of maximum total overtime shifts per nurse per week;
a number MO of maximum total overtime shifts per nurse per month.

Once the input values have been derived, a method 300 (FIG. 3A) is executed on the local computing device 20a (FIG. 2) by the scheduling algorithm 214 of the scheduling module 212. The scheduling algorithm includes an initialization stage 302 followed by a resource maximizing phase 300a followed by a load minimizing phase 300b. The initialization stage 302 includes setting n, m, θ, δ_{sd}, λ_{sd} and σ_{i,l} to 0 and γᵢₗ to M, wherein n and m are iteration counters, θ is a target load parameter used in deviation minimization, δ_{sd} is a binary parameter indicating whether a total shift allocation is maximized, λ_{sd} is a lower bound on a number of shifts assigned, σ_{i,l} is a binary parameter indicating whether a nurse load is minimized, γᵢₗ is an upper bound on a number of shifts assigned, and M is a sufficiently large number greater than 500 and preferably greater than 1000.

After the initialization stage 302, the algorithm continues to the resource maximizing phase, which includes: a first step 304a, 304b of determining (at 304a) if all available shifts have been maximized in which Σ_{s∈S}Σ_{d∈D}δ_{sd}=|S∥D| and if so, ending the maximizing phase, and if not, setting n=n+1 and proceeding to maximize all available shifts through solving an objective function P_{R}=αₙ (at 304b) using a constraint

$$( not shown ) : {\sum }_{i , l ∈ IL} {x}_{il}^{swd} + {y}_{il}^{swd} \geq {α}_{n} - M {δ}_{sd} for {P}_{R} ( θ , δ , λ , σ , γ ) \qquad ( 15 )$$

in which αₙ is a current level of shift allocation being maximized. The constraint

$${\sum }_{i , l ∈ IL} {x}_{il}^{swd} + {y}_{il}^{swd} \geq {α}_{n} - M {δ}_{sd} \qquad ( 15 )$$

ensures that shifts are fairly allocated and progressively maximized, and allows blocking shifts to be exempt from further maximization once they have reached their upper limits, thereby ensuring fairness across all shifts.

A second step 306 in the resource maximizing phase includes setting λ_{sd}=αₙ at iteration n to progressively increase shift assignments and prevent optimized shift allocations from being reduced in later iterations. A third step 308 includes setting θ=αₙ+ε, and solving an objective function

$${P}_{SND} = {\sum }_{s ∈ S} {\sum }_{d ∈ D} ( 1 - {δ}_{sd} ) {d}_{sd}^{-}$$

for P_{SND}(θ, δ, λ, σ, γ) to minimize a sum of negative deviations (P_{SND}) in which E is a number less than 1 and d_{sd}⁻ is a negative deviation from a total number of shifts for shift s on day d. A fourth step 310 includes setting δ_{sd}=1 if d_{sd}⁻ fails to be minimized to zero. Next, the first 304, second 306, third 308, and fourth 310 steps are repeated until all total shifts are maximized and cannot be increased further. The number of nurses assigned per day and shift is limited by the number MB of maximum beds per day per shift as defined by a constraint

$${\sum }_{i , l ∈ IL} {x}_{il}^{swd} + {y}_{il}^{swd} \leq MB ,$$

where x and y are binary decision variables, and once all shifts are maximized, proceeding to the load minimizing phase 300b.

The load minimizing phase 300b includes a fifth step 312a, 312b of determining (at 312a) if

$${\sum }_{i , l ∈ IL} {σ}_{il} = \vert I \vert \vert L \vert$$

and if not, setting (at 312b) m=m+1 and finding a minimum βₘ by solving an objective function P_{L}=βₘ (at 312b) for P_{L}(θ, δ, λ, σ, γ) where βₘ is an upper bound on a number of loads assigned to each nurse at iteration m. A sixth step 314 includes setting γᵢₗ=βₘ if σᵢₗ=0. An seventh step 316 includes setting θ=βₘ−ε and solving an objective function

$${P}_{SPD} = {\sum }_{( i , l ) ∈ IL} ( 1 - {σ}_{il} ) {d}_{il}^{+} for {P}_{SPD} ( θ , δ , λ , σ , γ )$$

to minimize a weighted sum of positive deviations (P_{SPD}) where \hat{d}ᵢₗ⁺ is a positive deviation from a total load of each nurse i of level l. An eighth step 318 includes determining if a load of a nurse i of level l is minimized by checking if dᵢₗ⁺>0 and if so, setting δᵢₗ=1 to designate the load is minimized. Next, the fifth (312a, 312b), sixth 314, seventh 316, and eighth 318 steps are repeated until all nurse loads are blocking. The method 300 concludes by converting the maximized resources and minimized loads into a completed schedule (at 320) to be distributed (at 322) to one or more remote computing devices.

FIG. 3B shows example sub-steps 320a-d to be executed by the scheduling module 212 (of FIG. 2) within step 320 of FIG. 3A, whereby the raw numerical output from the scheduling algorithm may be converted into a human-readable format such as a timetable or graphical schedule (shown in FIG. 3B). At 320a, the scheduling module extracts the output data from the scheduling algorithm, including which nurses are assigned to which shifts, the number of shifts each nurse is scheduled for, which shifts or workloads are blocking, and the final MMF shift distribution. At 320b, the data is converted into, for example, a structure table where rows represent nurses and columns represent shifts. Next, the structured data is formatted into, as non-limiting examples, a timetable at 320c or graphical schedule at 320d. To create a timetable (at 320c), a matrix is created by the scheduling module where, in a non-limiting example, the rows may list individual nurses, columns may list days, and cells contain may contain values such as “D” for Day, “N” for Night, or “O” for Off. To create a graphical schedule (at 320d), the process is similar to creating a timetable, but color coding/shading may be used, in a non-limiting example, to differentiate between shifts. For example: Day shifts(D)=first shade/color, Night shifts (N)=second shade/color, Off days (O)=third shade/color etc.

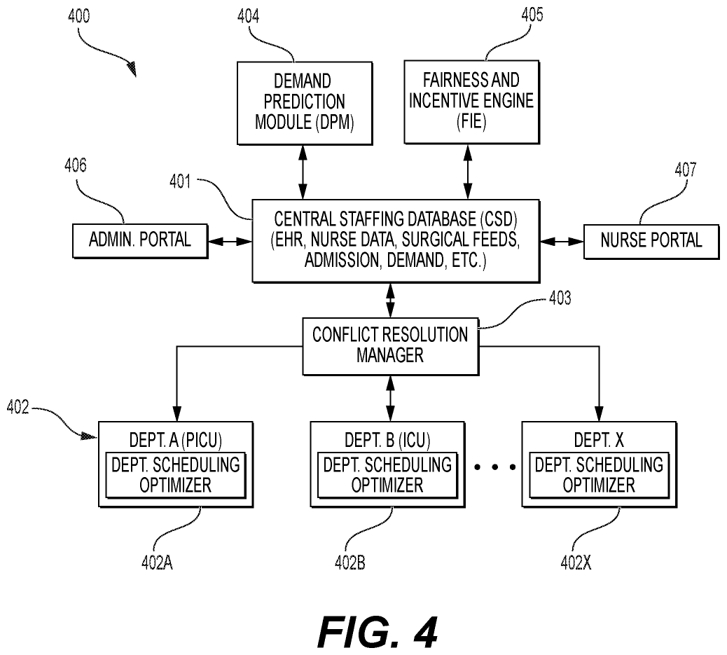

Referring now to FIG. 4, an embodiment of a distributed architecture of a dynamic nurse scheduling and optimization system is shown generally at 400. The system 400 includes a central staffing database (CSD) 401 in communication with a plurality of departmental scheduling optimizers (DSOs) 402, including, for example, DSO 402A associated with a first department, DSO 402B associated with a second department, and DSO 402X associated with one or more additional departments.

Each departmental scheduling optimizer 402A-X is implemented on one or more computing devices and is configured to generate and update nursing schedules for a corresponding department. In operation, each DSO 402A-X retrieves scheduling data from the central staffing database 401, performs localized schedule optimization, and transmits updated scheduling information back to the central staffing database 401. This distributed configuration enables parallel processing of scheduling computations across multiple departments while maintaining system-wide coordination.

The central staffing database 401 is configured to store and manage a plurality of data types required for scheduling operations, including, but not limited to, electronic health record (EHR) data, nurse profile and credential data, surgical scheduling data, hospital admission and discharge information, and predicted patient demand data. The central staffing database 401 may be implemented using one or more server systems and may maintain persistent storage of scheduling states, constraints, and optimization parameters, thereby enabling state preservation across iterative scheduling updates.

A demand prediction module 404 is communicatively coupled to the central staffing database 401 and is configured to receive data from one or more hospital information systems, including patient admission systems, surgical scheduling systems, and clinical reporting systems. The demand prediction module 404 processes such inputs to generate predicted demand metrics corresponding to future time intervals. These predicted demand metrics are transmitted to the central staffing database 401 and made available to each departmental scheduling optimizer 402 to guide scheduling decisions. In this manner, scheduling updates may be triggered in an event-driven fashion based on detected changes in patient demand.

A fairness and incentive engine 405 is also communicatively coupled to the central staffing database 401. The fairness and incentive engine 405 is configured to maintain and update fairness-related metrics associated with individual nurses, including participation levels and reward points. In one aspect, the fairness and incentive engine 405 dynamically adjusts incentive values associated with schedule modification requests in order to encourage voluntary acceptance of additional shifts or schedule changes while maintaining equitable workload distribution across available staff.

The system 400 further includes a conflict resolution manager 403 in communication with the central staffing database 401 and the plurality of departmental scheduling optimizers 402. The conflict resolution manager 403 is configured to detect conflicts arising from concurrent schedule updates, particularly where multiple departmental scheduling optimizers 402 attempt to allocate the same personnel resources. Upon detection of such conflicts, the conflict resolution manager 403 applies arbitration logic to resolve inconsistencies. In some embodiments, such arbitration logic may consider factors including, but not limited to, scheduling priority, fairness metrics, and personnel qualifications.

An administrative portal 406 and a nurse portal 407 are each communicatively coupled to the central staffing database 401. The administrative portal 406 may be accessed by hospital administrators or supervisory personnel to input scheduling requirements, approve cross-department staffing adjustments, and monitor system performance. The nurse portal 407 provides an interface through which nursing staff may view assigned schedules, receive scheduling offers, and accept or reject proposed modifications. Interactions provided through the administrative portal 406 and nurse portal 407 may trigger updates to scheduling inputs stored within the central staffing database 401, thereby initiating incremental reoptimization processes performed by one or more departmental scheduling optimizers 402.

In operation, the distributed architecture 400 enables real-time or near real-time schedule updates by distributing optimization tasks across the plurality of departmental scheduling optimizers 402 and synchronizing results through the central staffing database 401. When a change in system state is detected, such as an increase in patient demand, a staff availability update, or a user-initiated schedule modification, only affected portions of the schedule are recomputed by one or more departmental scheduling optimizers 402 rather than recomputing an entire scheduling horizon. This incremental recomputation reduces computational overhead and improves system responsiveness.

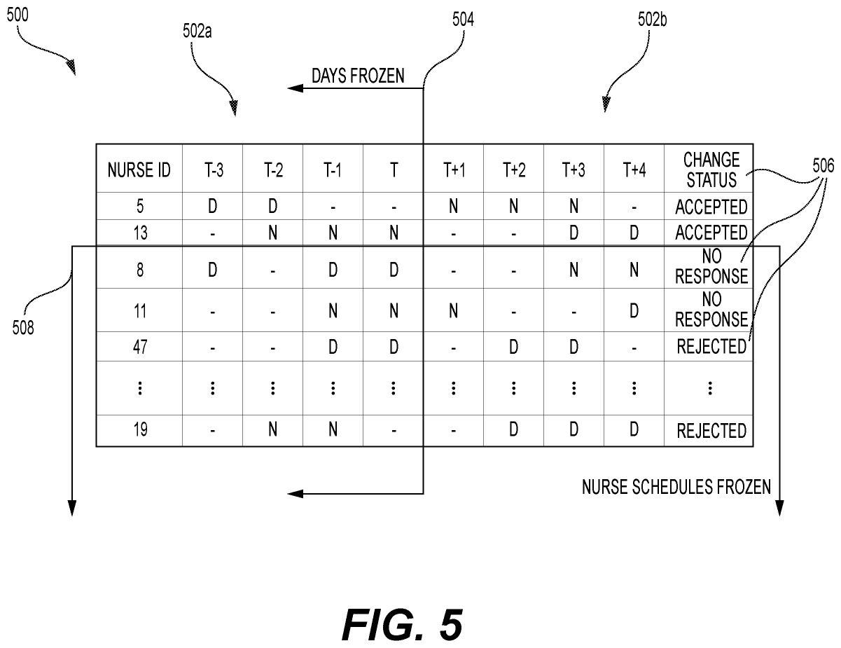

Referring now to FIG. 5, an example nurse scheduling timeline is shown generally at 500. The scheduling timeline 500 represents a rolling time window including past, present, and future scheduling intervals for a plurality of nurses. The timeline is divided into a first portion 502a corresponding to past scheduling periods and a second portion 502b corresponding to future scheduling periods.

A boundary 504 at or near a current time indicator separates “frozen” schedule segments from “modifiable” schedule segments. The frozen schedule segments correspond to time intervals for which scheduling decisions have been finalized and are not subject to further modification. Such frozen segments may include completed shifts as well as near-term scheduled shifts that have been locked to ensure operational stability.

The modifiable schedule segments correspond to future time intervals for which schedule updates may be applied. Within the modifiable region, individual schedule entries may be selectively updated based on system inputs, including predicted demand changes, staff availability, and responses received through the nurse portal 407 or administrative portal 406. In this regard, the system enables targeted adjustments to scheduling assignments without requiring complete regeneration of the entire schedule.

The scheduling timeline 500 further illustrates a set of nurse-specific schedule entries 506, each associated with a corresponding status indicator reflecting whether a proposed schedule modification has been accepted, rejected, or not responded to. In certain embodiments, schedule entries corresponding to accepted modifications may be incorporated into subsequent optimization cycles, while schedule entries corresponding to rejected or non-responsive actions may be preserved as frozen entries to prevent repeated reassignment attempts, as indicated by the boundary 508.

In this manner, the scheduling timeline 500 enables incremental, event-driven schedule updates in which only selected portions of the schedule are modified based on user interaction and system inputs while previously finalized schedule entries are maintained. This selective update mechanism improves computational efficiency and reduces disruption to existing schedules by limiting the scope of recalculation to affected time intervals and personnel.

### Experimentation and Testing

During development of the subject matter of the present application, a pediatric intensive care unit (PICU) was investigated at a large local Saudi hospital. The PICU unit admits children over the age of one month up to 12 years old for cases that require intensive care such as acute infections, heart stimulants, kidney and liver dialysis, in addition to cases of burns and major surgical cases. The PICU bed capacity was 21, and the total number of available nurses was 66 with some nurses being senior level nurses. Assignments were made of one nurse to one patient.

The schedule was built every 4 weeks (28 days), and was considered a monthly schedule. Nurses were required to work two 12-hour shifts (day and night). Each nurse was required to work 48 hours per week as regular time, and a maximum of 72 hours per week including overtime. These requirements translate to a minimum of 4 regular shifts per week, a maximum of 2 overtime shifts per week, and a maximum of 5 overtime shifts per month. The scheduling department builds a 28-day schedule every four weeks for which it is preferable if a nurse works day shifts for two weeks followed by night shifts for two weeks or the opposite. A mandatory requirement is that the nurse takes at least two days off when switching from a night shift to a day shift or the opposite. In addition, a nurse may not work more than four consecutive days. Otherwise, the nurse must take at least one day off. Moreover, in each shift, a team leader or an in-charge (i.e. a senior level) nurse must be present to supervise the staff.

The hospital approach in scheduling was to schedule all the nurses and all the overtime shifts even if they are not all needed. After which, the hospital starts utilizing or canceling the overtime shifts based on the demand since the demand in the unit is unstable. If the hospital cancels all overtime shifts and still has excess nurses, the nurses are distributed to other departments needing assistance. Once the hospital utilizes all overtime shifts and are still short in staffing, some nurses are assigned to double beds. Hence, in the experimental study, a similar approach was followed to produce an optimized schedule with full capacity to be prepared for any unexpected demand.

### The DDPFA Algorithm

| Algorithm: | Finds MMF resource allocation |
|---|---|
| Initialization | Set n = 0, m = 0, θ = 0, δ_{sd} = 0, and λ_{sd} = 0 ∀ s ∈ S, |
| | d ∈ D, σ_{i,l} = 0, and γᵢₗ = M ∀ (i, l) ∈ IL |
| Step 1 | $If {\sum }_{s ∈ S} {\sum }_{i ∈ I} {δ}_{sd} = \vert S \vert \vert D \vert , go to step 7. Otherwise , set n =$ |
| | n + 1 and find the maximum αₙ by solving the problem |
| | P_{R}(θ, δ, λ, σ, γ) |
| Step 2 | If δ_{sd} = 0, set λ_{sd} = αₙ, and ∀ s ∈ S, d ∈ D |
| Step 3 | Set θ = αₙ + ε, and solve the problem P_{SND} (θ, δ, λ, σ, γ) |
| | Set θ = αₙ + ε, and solve P_{SND} (θ, δ, λ, σ, γ) |
| Step 4 | $If {d}_{sd}^{-} > 0 , set {δ}_{sd} = 1 ∀ s ∈ S , d ∈ D$ |
| Check - all | No - Go to step 2 |
| resources | |
| blocking? | Yes - Go to step 5 |
| Step 5 | $If {\sum }_{i , l ∈ IL} {σ}_{il} = \vert I \vert \vert L \vert , go to step 12. Otherwise , set n =$ |
| | m + 1 and find the minimum βₘ by solving the problem |
| | P_{L}(θ, δ, λ, σ, γ) |
| Step 6 | If σᵢₗ = 0, set γᵢₗ = βₘ, and ∀ (i, l) ∈ IL |
| Step 7 | $Set θ = {β}_{m} - ε , and solve the problem {P}_{SPD} ( θ , δ , λ , σ , γ )$ |
| | $Set θ = {β}_{m} - ε , and solve {P}_{SPD} ( θ , δ , λ , σ , γ )$ |
| Step 8 | $If {d}_{il}^{+} > 0 , set {δ}_{il} = 1 ∀ ( i , l ) ∈ IL$ |
| Check - all | No - Go to step 5 |
| loads | |
| blocking? | Yes - Proceed to generate completed schedule |

During experimentation, the DDPFA algorithm was implemented in two scenarios. In the first scenario, all 66 nurses were assumed to be available over the 28-day scheduling horizon. In the second scenario, a real case scenario was experimented where some nurses are not available due to being on leave. The MMF nurse schedule is compared with schedules optimized with different objectives namely maximizing the total resources and maximizing the minimum number of resources.

For comparison purposes, different objective functions were tested to highlight the improvements gained by finding the MMF nurse schedule using the DDPFA algorithm. The objective of the first comparison model:

$$Max TR = {\sum }_{( i , l ) ∈ IL} {\sum }_{s ∈ S} {\sum }_{( w , d ) ∈ WD} {x}_{il}^{swd} + {y}_{il}^{swd}$$

is maximizing the total resources (TR) by maximizing the total regular and overtime working shifts for all nurses, all days, in all shifts, subject to the previous constraints (5)-(14).

The second comparison model was to maximize the minimum resources (MR) bound r and minimize the maximum load (ML) ω, solved in two phases. In the first phase, the objective function: Max MR=τ is maximized using the constraint:

$${\sum }_{( i , l ) ∈ IL} {x}_{il}^{swd} + {y}_{il}^{swd} \geq τ$$

and the previous set of constraints (5)-(14). The optimal r found in the first phase is fed into the second phase through the constraint:

$${\sum }_{i , l ∈ IL} {x}_{il}^{swd} + {y}_{il}^{swd} \geq τ .$$

The second objective function: Min ML=ω is used to minimize the maximum load for all nurses imposing constraint:

$${\sum }_{s ∈ S} {\sum }_{d ∈ D} {x}_{il}^{swd} + {y}_{il}^{swd} \leq ω$$

and the previous set of constraints (5)-(14).

### Results

The results compare the total resources (total regular and overtime shifts), and the total loads (assigned shifts per nurse) resulting from the three models presented. All three models were implemented under two scenarios. In the first scenario, all 66 nurses are available during the 28 days, and in the second scenario some nurses are unavailable due to being on leave.

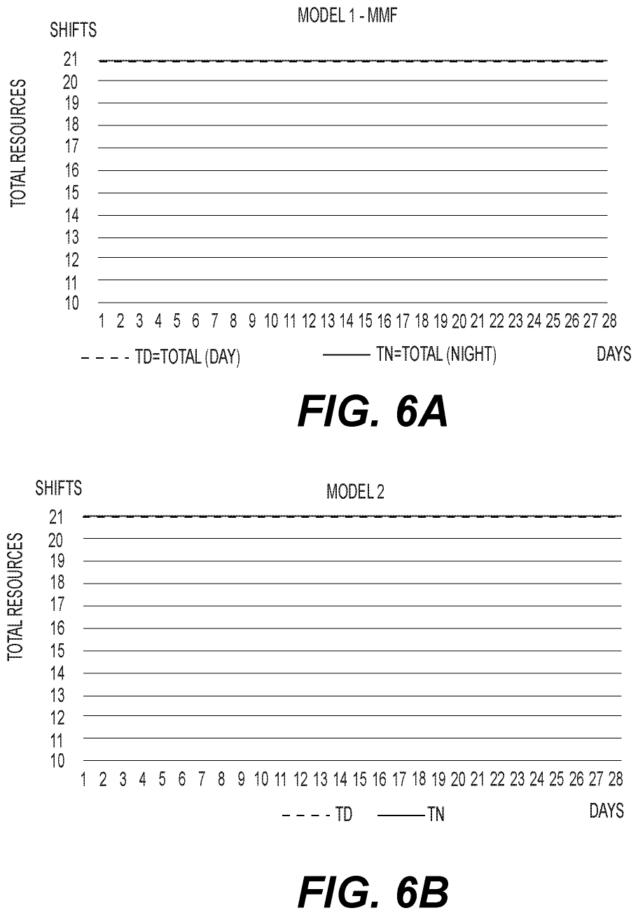

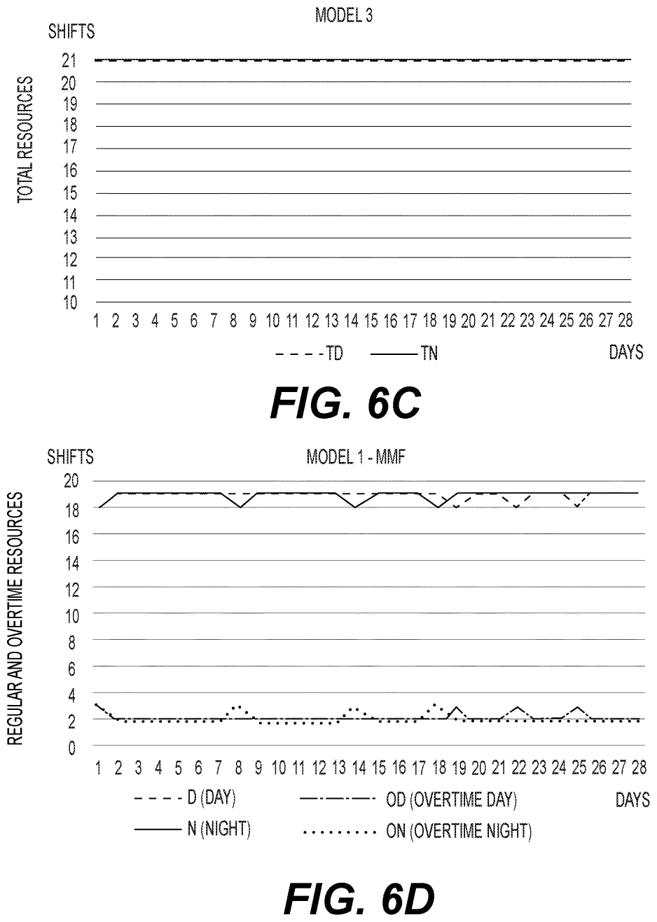

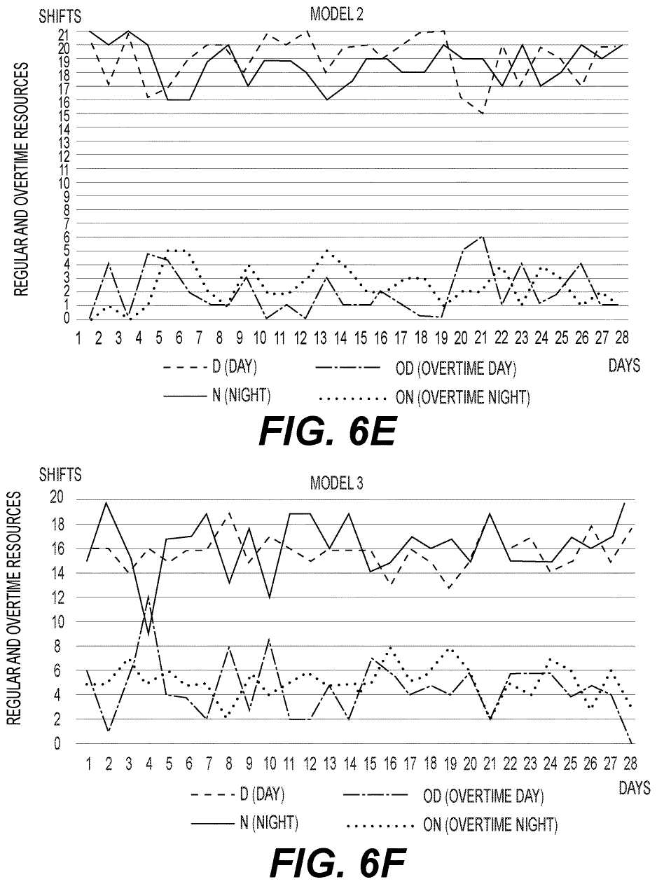

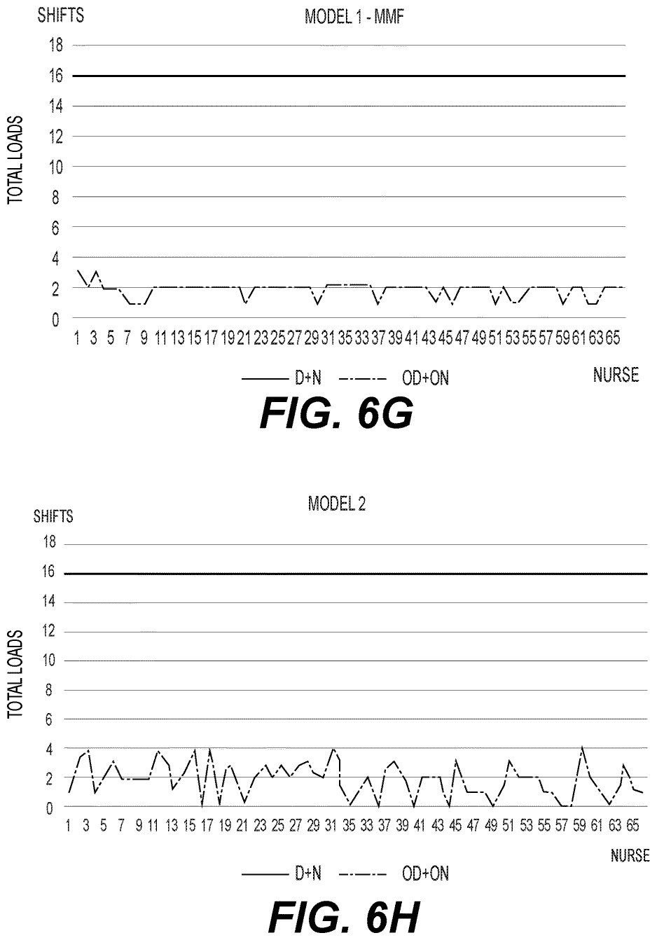

FIGS. 6A-I, and 7A-I illustrate example performance characteristics and scheduling outcomes generated by the scheduling algorithm. In certain embodiments, the scheduling algorithm may operate without weighting parameters, while in other embodiments, weighting factors such as shift urgency or nurse fairness weights may be incorporated to further refine scheduling outcomes. Accordingly, the illustrated results represent example behaviors of the scheduling framework and are not limiting of implementations incorporating additional weighting parameters. In embodiments incorporating weighting parameters, including urgency weights and fairness weights, similar scheduling behaviors may be observed, with deviations adjusted according to demand sensitivity and workload prioritization.

FIGS. 6A-1 show the results of the three models under scenario 1 where the assumption is that all 66 nurses are available for all 28 days. As shown in FIGS. 4A-I, the total resources reached their maximum (21 beds) in all three models as seen in FIGS. 4A, 4 B, and 4C since the available nurses are able to cover all the demand. However, the regular and overtime resources in both models 2 and 3 were shown to be unstable in the charts of FIG. 4E and FIG. 4F. However, the regular and overtime resources are shown to be more stable for model 1 in the chart of FIG. 4D, which allows for more flexibility in removing overtime shifts when not needed to follow a similar policy to the PICU. On the other hand, the loads assigned to each nurse are investigated as seen in the charts of FIGS. 4G, 4H, and 4I. Note that all nurses are fairly assigned 16 regular working shifts when using model 1, but regular shifts are not sufficient to cover all the demands. Hence, all nurses are assigned 1 to 2 overtime shifts (except nurses 1 and 3 who are assigned 3 overtime shifts). One might argue that overtime shifts should be transferred from nurses 1 and 2 to other nurses who are assigned one overtime shift, which might not be possible because the way the load is distributed puts nurses in a position where they cannot take any more load, and the load resulting from model 1 is distributed based on maximum-minimum fairness (MMF). Note that the load is highly varied among nurses in models 2 and 3 as shown in the charts of FIGS. 4H and 4I.

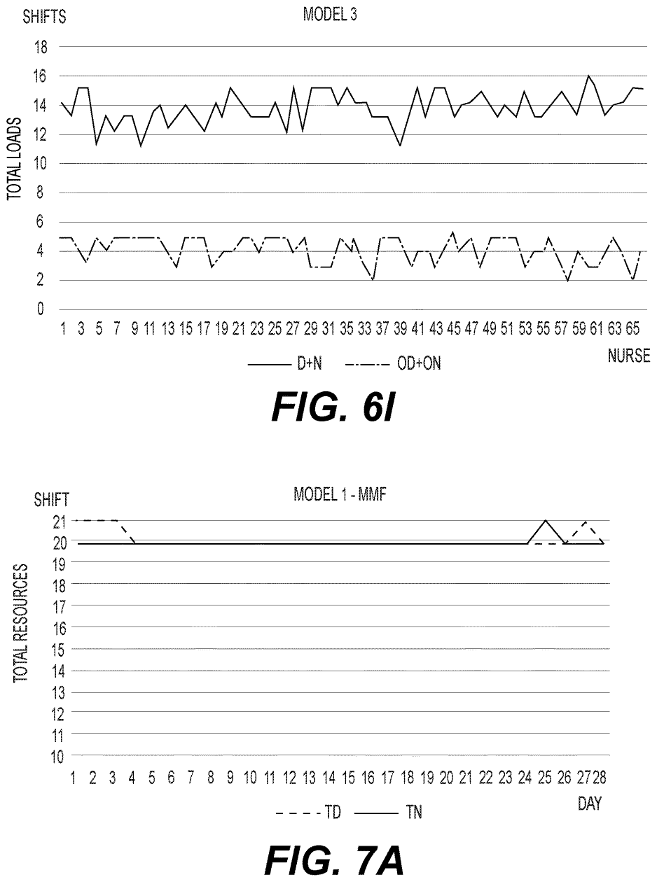

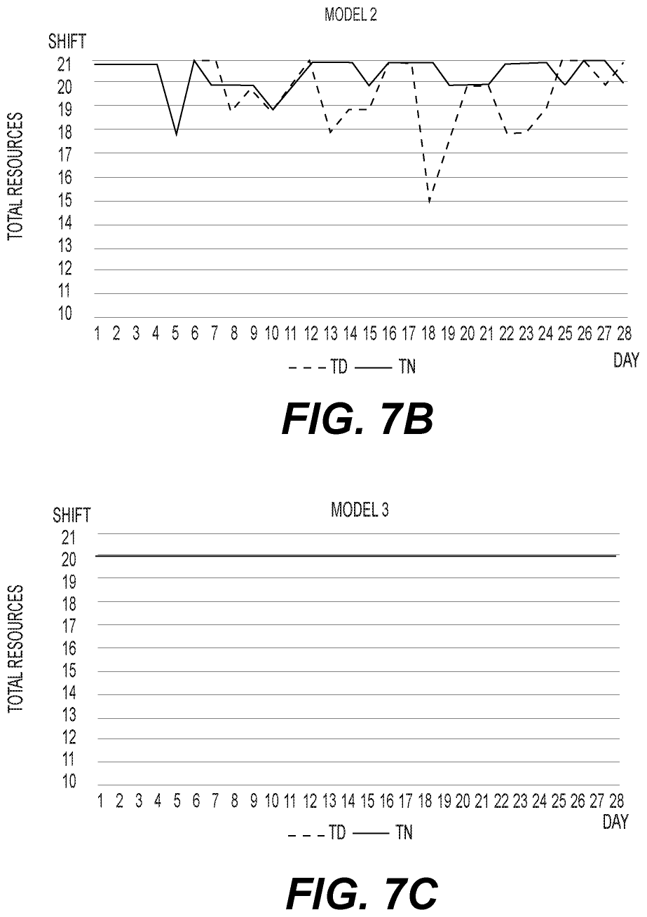

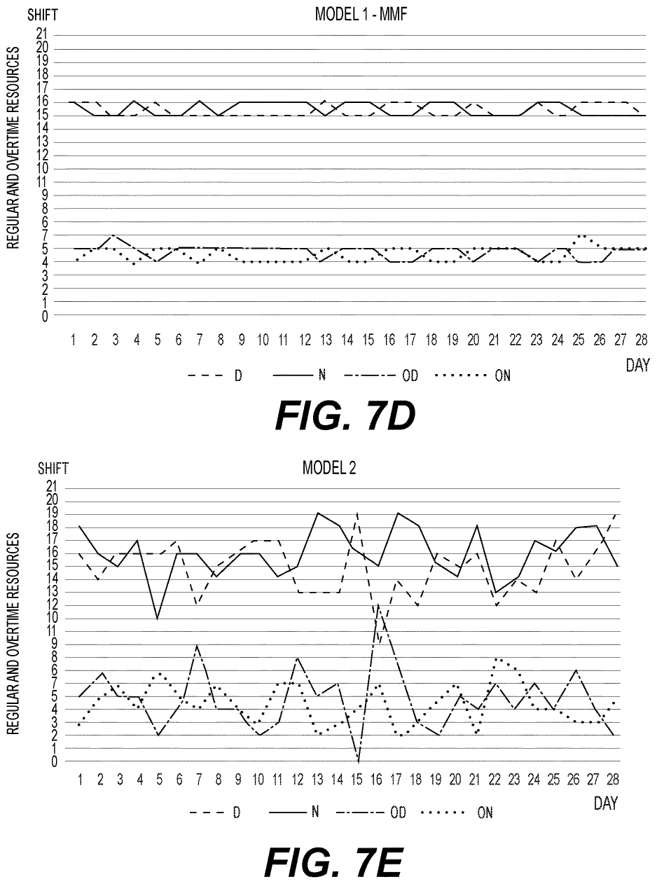

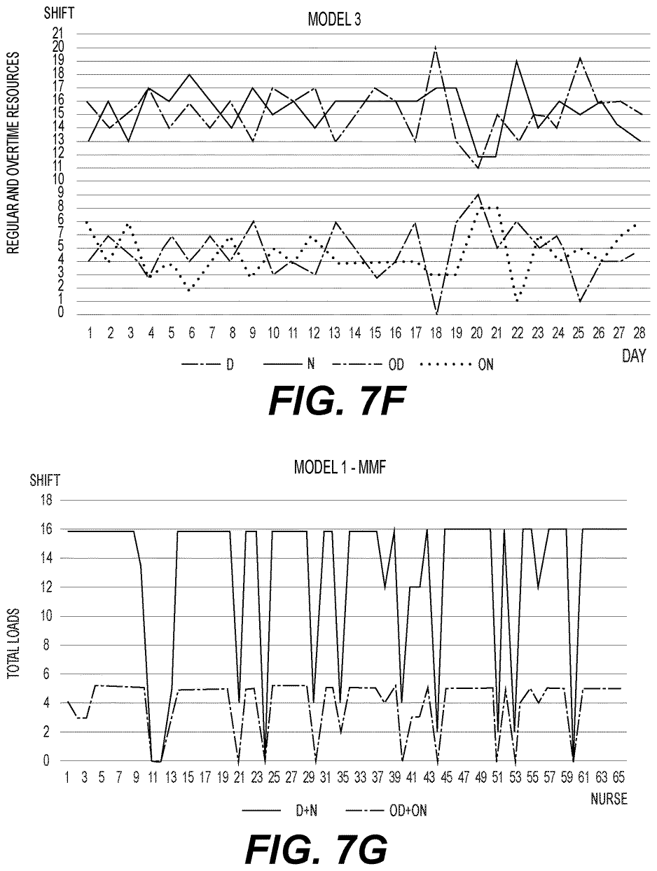

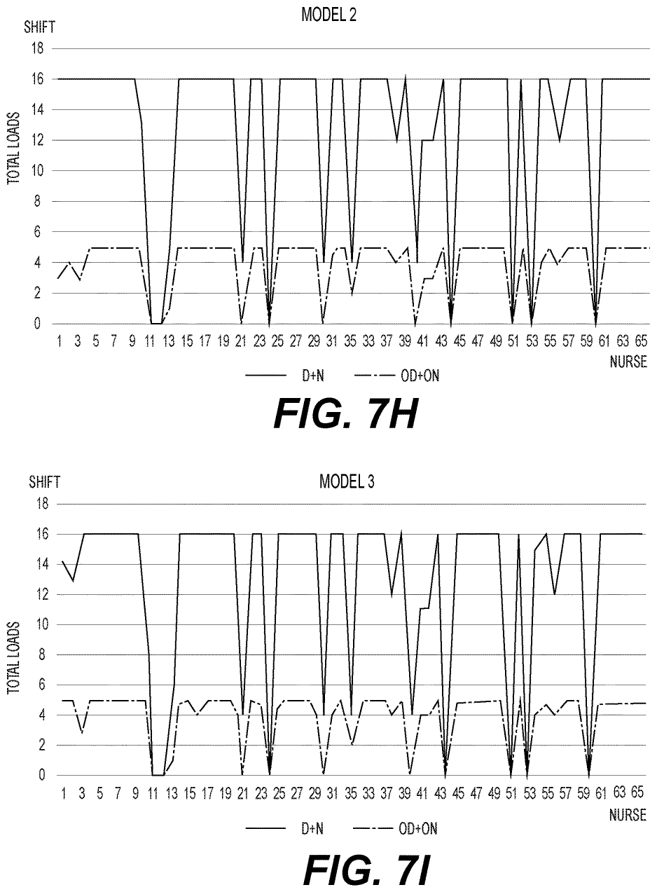

FIGS. 7A-I show the results of the three models under scenario 2 where the assumption is that some nurses are not available due to being on leave. The total resources achieved for each day and shift is based on maximum-minimum fairness (MMF) which has been reached in two iterations as illustrated in the chart of FIG. 5A. In the first iteration, the minimum bound on the number of resources is maximized to 20, then the blocking resources are removed from the maximization process to proceed with the non-blocking resources to reach 21 shifts in the second iteration. Not all beds are assigned to nurses due to the shortage of nurses. The total resources resulting from model 3 are similar to the results of model 1 but still did not utilize all the available resources. Hence, model 1 outperformed model 3 in utilization as seen in the chart of FIG. 5C. Model 2 was able to reach the maximum utilization, but failed to distribute the resources among days and shifts evenly.

Regarding the even distribution of regular and overtime resources among days and shifts, model 1 clearly outperforms models 2 and 3 as shown in the respective charts of FIGS. 5D, 5E, and 5F. The total loads are similar for all models as seen in the charts of FIGS. 5G, 5H, and 5I because all nurses have reached their maximum capacity to cover the shortage of nurses. Hence, there is no room for fairness in this scenario.

TABLE 1
Summary of the results for both scenarios
experimenting the three models

| | Scenario | | | | | |
|---|---|---|---|---|---|---|
| | 1 | | | 2 | | |
| | Model | | | | | |
| | 1 | 2 | 3 | 1 | 2 | 3 |
| Objective 1 | 3 | 1 | 1 | 4 | 1 | 1 |
| iterations (n) | | | | | | |
| Objective 2 | 6 | — | 1 | 13 | — | 1 |
| iterations (m) | | | | | | |
| Assigned beds | 1176 | 1176 | 1176 | 1125 | 1125 | 1120 |
| Unassigned | 0 | 0 | 0 | 51 | 51 | 56 |
| beds | | | | | | |
| Total regular | 1056 | 1056 | 901 | 864 | 864 | 856 |
| shifts | | | | | | |
| Total overtime | 120 | 120 | 275 | 261 | 261 | 264 |
| shifts | | | | | | |
| Total resources | 0 | 0 | 0 | 0.08 | 1.47 | 0 |
| variance | | | | | | |
| Regular | 0.12 | 2.62 | 4.29 | 0.24 | 4.53 | 3.31 |
| resources | | | | | | |
| variance | | | | | | |
| Overtime | 0.28 | 2.62 | 4.29 | 0.30 | 4.26 | 3.31 |
| resource | | | | | | |
| variance | | | | | | |
| Regular load | 0 | 0 | 1.26 | 30.81 | 30.81 | 30.57 |
| variance | | | | | | |
| Overtime load | 0.32 | 1.42 | 0.84 | 3.38 | 3.38 | 3.33 |
| variance | | | | | | |

The summary of the results is reported in Table 1. Note that model 1 (the proposed model) requires multiple iterations to find the MMF solution for both resources and loads. Model 1 is solved using one iteration since it only uses one objective function, and model 2 uses two objective functions. Note that all beds are assigned to nurses in scenario 1 since the assumption is that all nurses are available. However, about 5% of beds are unattended in some shifts because some nurses are on leave. If these beds are occupied, some nurses would be double assigned to beds in these specific shifts. Note that models 1 and 2 are identical in terms of bed utilization for both scenarios because the proposed model 1 achieves high utilization when pursuing fairness. Also, the number of regular and overtime shifts is identical in models 1 and 2 in both scenarios. On the other hand, model 3 does not prioritize the regular shifts. Hence, it assigns more overtime shifts compared to models 1 and 2.

Next, a comparison is implemented in terms of fair distribution of resources and loads through measuring variance since it measures how much the number of shifts deviates from the mean. In scenario 1, the variance is 0 for the three models. However, in scenario 2, model 3 outperforms models 1 and 2 in terms of fairness which is possible because it has a lower utilization of beds as model 3 accounts for fairness, and model 1 accounts for fairness and utilization.

In both scenarios, the reduction in variance almost exceeds 90% in the number of regular and overtime resources when comparing model 1 with models 2 and 3. The load variance of model 1 is either equal or superior to models 2 and 3 in scenario 1. Nevertheless, the variance of the loads is similar in scenario 2 for models 1 and 2 because all nurses are assigned their maximum capacity. In model 3, the load variance is slightly lower because the nurses are not fully utilized.

The present disclosure provides a method of allocating work shifts fairly among nurses using a maximum-minimum fairness (MMF) approach. The method operates in two primary phases, first, by maximizing shift assignments across available resources (nurses) until certain constraints, known as blocking resources, prevent further increases. In the second phase, the method minimizes individual nurse workloads, ensuring that no nurse is disproportionately overburdened. By iteratively adjusting shift distributions and enforcing fairness constraints, the method balances demand while prioritizing equity. The final schedule ensures that shifts and workloads are fairly distributed, considering nurse preferences, hospital requirements, and workload fairness.

It is to be understood that the method for generating a nursing schedule with maximum-minimum fairness is not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

1. A method for generating a nursing schedule, comprising:
installing a scheduling module onto a local computing device communicatively coupled over a network to a central staffing database and a plurality of remote computing devices;
receiving, on the local computing device, hospital division requirements, nurse availability, and nurse preferences including real-time operational data comprising at least one of patient admission data, discharge data, or clinical event data received from one or more hospital information systems, to derive input values including:
a set I of nurses, wherein values i in the set of nurses include names of individual nurses;
a set L of nurse levels associated with the set of nurses, wherein the values l in the set of nurse levels indicate if a nurse is a senior level nurse;
a two-dimensional set IL of each nurse and their level;
a set of preferences associated with the set of nurses, wherein the set of preferences associated with the set of nurses include shift preferences of weekday, weekend, day, and night;
a set W of weeks, wherein values w in the set of weeks include week-long time periods on a calendar;
a set D of days, wherein values in the set of days indicate different days d of a week;
a two-dimensional set WD of weeks and days;
a set S of shifts per day, wherein the set of shifts per day include a number of shifts s required for a calendar day;
a number MB of maximum beds per day per shift;
a number WR of maximum regular shifts per nurse per week;
a number MR of maximum total regular shifts per nurse per month;
a number WO of maximum total overtime shifts per nurse per week;
a number MO of maximum total overtime shifts per nurse per month;
executing on the local computing device a scheduling algorithm through the scheduling module, wherein the scheduling module is configured to perform event-driven and incremental schedule recomputation in response to detected changes in said real-time operational data, wherein the scheduling algorithm includes an initialization stage followed by a resource maximizing phase followed by a load minimizing phase;
wherein the initialization stage includes setting n, m, θ, δ_{sd}, λ_{sd} and σ_{i,l} to 0 and γᵢₗ to M, wherein n and m are iteration counters, θ is a target load parameter used in deviation minimization, δ_{sd} is a binary parameter indicating whether a total shift allocation is maximized, λ_{sd} is a lower bound on a number of shifts assigned, σ_{i,l} is a binary parameter indicating whether a nurse load is minimized, γᵢₗ is an upper bound on a number of shifts assigned, and M is a large number greater than 500 and preferably greater than 1000;
the resource maximizing phase including:
a first step of determining if all available shifts have been maximized in which
$${\sum }_{s ∈ S} {\sum }_{i ∈ I} {δ}_{sd} = \vert S \vert \vert D \vert$$
and if so, ending the maximizing phase, and if not, setting n=n+1 and proceeding to maximize all available shifts through solving an objective function P_{R}=αₙ using constraint
$${\sum }_{i , l ∈ IL} {x}_{il}^{swd} + {y}_{il}^{swd} \geq {α}_{n} - M {δ}_{sd} for {P}_{R} ( θ , δ , λ , σ , γ )$$
in which αₙ is a current level of shift allocation being maximized;
a second step of setting λ_{sd}=αₙ at iteration n to progressively increase shift assignments and prevent optimized shift allocations from being reduced in later iterations;
a third step of setting θ=αₙ+ε, and solving an objective function
$${P}_{SND} = {\sum }_{s ∈ S} {\sum }_{d ∈ D} ( 1 - {δ}_{sd} ) {U}_{sd} {d}_{sd}^{-} for {P}_{SND} ( θ , δ , λ , σ , γ )$$
to minimize a sum of negative deviations (P_{SND}) in which ε is a number less than 1 and preferably less than 0.6 and d_{sd}⁻ is a negative deviation from a total number of shifts for shift s on day d and U_{sd} is a parameter representing the weight of the shift s on day d;
a fourth step of setting
$${δ}_{sd} = 1 if {d}_{sd}^{-}$$
fails to be minimized to zero;
repeating the first, second, third, and fourth steps until all total shifts are blocking, wherein a number of nurses assigned per day and shift is limited by the number MB of maximum beds per day per shift as defined by a constrain
$${\sum }_{i , l ∈ IL} {x}_{il}^{swd} + {y}_{il}^{swd} \leq MB$$
where x and y are binary decision variables, and once all shifts are blocking, proceeding to the load minimizing phase, wherein the load minimizing phase includes:
a fifth step of determining if
$${\sum }_{i , l ∈ IL} {σ}_{il} = \vert I \vert \vert L \vert$$
and if not, setting m=m+1 and finding a minimum βₘ by solving an objective function P_{L}=βₘ for P_{L}(θ, δ, λ, σ, γ) where βₘ is an upper bound on a number of loads assigned to each nurse at iteration m;
a sixth step of setting γᵢₗ=βₘ if σᵢₗ=0;
a seventh step of setting θ=βₘ−ε and solving an objective function
$${P}_{SPD} = {\sum }_{( i , l ) ∈ IL} ( 1 - {σ}_{il} ) {P}_{il} {d}_{il}^{+} for {P}_{SPD} ( θ , δ , λ , σ , γ )$$
to minimize a weighted sum of positive deviations (P_{SPD}) where \hat{d}ᵢₗ⁺ is a positive deviation from a total load of each nurse i of level l and Pᵢₗ is a parameter representing the weight of the nurse i of level l;
an eighth step of determining if a load of a nurse i of level l is minimized by checking if
$${d}_{il}^{+} > 0$$
and if so, setting δᵢₗ=1 to designate the load is minimized;
repeating the fifth, sixth, seventh, and eighth steps until all nurse loads are blocking;
updating the central staffing database with the recomputed schedule while synchronizing schedule changes with one or more remote computing devices in substantially real time;
converting resulting numerical data into a human-readable completed schedule by:
extracting, by the scheduling module, numerical shift allocation data generated by the scheduling algorithm;
converting the numerical shift allocation data into a structured schedule matrix stored in memory of the local computing device
automatically generating, from the structured scheduled matrix, a machine-generated timetable representing nurse shift assignments; and
electronically sending the completed schedule from the local computing device to one or more remote computing devices.

2. The method for generating a nursing schedule according to claim 1, wherein a constraint
$${\sum }_{s ∈ S} {\sum }_{d ∈ D} {x}_{il}^{swd} \leq MR$$
ensures that each nurse does not exceed the number MR of maximum total regular shifts per nurse per month.

3. The method for generating a nursing schedule according to claim 1, wherein a constraint
$${\sum }_{s ∈ S} {\sum }_{d ∈ D} {y}_{i l}^{s w d} \leq MO$$
ensures that each nurse does not exceed the number MO of maximum total overtime shifts per nurse per month.

4. The method for generating a nursing schedule according to claim 1, wherein a constraint
$${\sum }_{d \vert ( w , d ) ∈ W D} {\sum }_{s ∈ S} {x}_{i l}^{s w d} \leq W S$$
ensures that each nurse does not exceed a number WS of regular and overtime shifts each week, wherein the number WS of regular and overtime shifts each week is derived from the hospital division requirements.

5. The method for generating a nursing schedule according to claim 1, wherein a constraint
$$1 - {x}_{i l}^{2 - a , wd} - {y}_{i l}^{2 - a , wd} \geq {x}_{i l}^{1 + a , wd + b} + {y}_{i l}^{1 + a , wd + b}$$
prevents switching a nurse from a day shift to a night shift until the nurse takes two days off given that parameter a switches shifts, and parameter b applies the constraint on the next day.

6. The method for generating a nursing schedule according to claim 1, wherein a constraint
$${\sum }_{s ∈ S} {x}_{i l}^{s w d} + {y}_{i l}^{s w d} \leq 1$$
prevents assigning a regular shift and an overtime shift to each nurse simultaneously.

7. The method for generating a nursing schedule according to claim 1, wherein a constraint
$$4 - {\sum }_{k = d}^{d + 4} {\sum }_{s ∈ S} {x}_{i l}^{swk} + {y}_{i l}^{swk} \geq {x}_{i l}^{swd + 4} + {y}_{i l}^{swd + 4}$$
sets a limit on a number of consecutive working days to be four days, after which a nurse is required to take at least one day off.

8. The method for generating a nursing schedule according to claim 1, wherein a constraint
$${x}_{i l}^{swd} + {y}_{i l}^{swd} \geq 1$$
assigns at least one senior level nurse in every shift.

9. The method for generating a nursing schedule according to claim 1, wherein a constraint
$${\sum }_{s ∈ S} {x}_{i l}^{swd} + {y}_{i l}^{swd} \leq {A}_{id}$$
stops assigning any load to a nurse i if they are not available, in which A_{id} is a parameter that equals 1 if a nurse i is available to work on day d and 0 otherwise.

10. The method for generating a nursing schedule according to claim 1, wherein a constraint
$${\sum }_{i , l ∈ l L} {x}_{i l}^{swd} + {y}_{i l}^{swd} - θ + {d}_{swd}^{-} - {d}_{swd}^{+} = 0$$
is used to ensure that a total number of assigned shifts for nurse i on a day d of week w remains as close as possible to the target load parameter θ.

11. The method for generating a nursing schedule according to claim 1, wherein a constraint
$${\sum }_{i , l ∈ lL} {U}_{s d} ( {x}_{i l}^{swd} + {y}_{i l}^{swd} ) - {λ}_{swd} \geq 0$$
sets a lower boundary for shifts.

12. The method for generating a nursing schedule according to claim 1, wherein a constraint
$${\sum }_{s ∈ S} {\sum }_{d \vert ( w , d ) ∈ WD} {P}_{il} ( {x}_{i l}^{s w d} + {y}_{i l}^{s w d} ) \leq {β}_{n} + M {σ}_{i l}$$
dynamically regulates how many shifts a nurse can be assigned, enforcing fairness until loads reach a blocking state where the loads can no longer be reduced.

13. The method for generating a nursing schedule according to claim 1, wherein a constraint
$${\sum }_{s ∈ s} {\sum }_{d \vert ( w , d ) ∈ WD} {x}_{i l}^{s w d} + {y}_{il}^{swd} - θ + {\hat{d}}_{i l}^{-} - {\hat{d}}_{i l}^{+} = 0$$
enforces fairness by ensuring each nurse i is assigned a workload close to the target parameter θ, allowing for small positive or negative deviations to maintain feasibility in scheduling.

14. The method for generating a nursing schedule according to claim 1, wherein a constraint
$${\sum }_{s ∈ S} {\sum }_{d \vert ( w , d ) ∈ WD} {P}_{il} ( {x}_{i l}^{s w d} + {y}_{i l}^{s w d} ) - {γ}_{i l} \leq 0$$
prevents any nurse from being overburdened by limiting total assigned shifts to be no greater than γᵢₗ.

15. The method for generating a nursing schedule according to claim 1, wherein a constraint
$${x}_{i l}^{s w d} , {y}_{i l}^{s w d} ∈ \{ 0 , 1 \}$$
enforces a binary yes or no decision for assigning nurses to shifts, ensuring that partial assignments are not allowed.

16. The method for generating a nursing schedule according to claim 1, wherein a constraint
$${d}_{s w d}^{-} , {d}_{s w d}^{+} \geq 0$$
ensures that imbalances in shift assignments are measured as a non-negative deviation thereby preventing cancellation effects where an excess of shifts offsets a shortage of shifts.

17. The method for generating a nursing schedule according to claim 1, wherein a constraint
$${\hat{d}}_{il}^{-} , {\hat{d}}_{i l}^{+} \geq 0$$
is used to track and correct any unfair distribution of shifts among nurses by ensuring deviations are positive whereby imbalances can be quantified and minimized, ensuring that no nurse is overburdened or underutilized unfairly.

18. The method for generating a nursing schedule according to claim 1, wherein hospital division requirements include bed capacity, nurse shift length, required work hours per week, maximum overtime hours per week, and total days and weeks of schedule.

19. The method for generating a nursing schedule according to claim 1, wherein the completed schedule is in the form of a timetable comprising rows and columns.

20. The method for generating a nursing schedule according to claim 1, wherein the completed schedule is in the form of a timetable comprising color-coding or shading, wherein the color-coding or shading indicates shift assignments for nurses.
